(19) United States
(12) Patent Application Publication
(10) Pub. No.: US 2013/0022673 A1
(43) Pub. Date: Jan. 24, 2013

(54) ENHANCEMENT OF MAGNESIUM UPTAKE IN MAMMALS

(71) Applicant: Russell Jaffe, Vienna, VA (US)

(72) Inventor: Russell Jaffe, Vienna, VA (US)

(21) Appl. No.: 13/627,530

(22) Filed: Sep. 26, 2012

Related U.S. Application Data
(63) Continuation-in-part of application No. 13/082,939, filed on Apr. 8, 2011, now abandoned.
Application No. 13/082,939 is a continuation of application No. 10/641,291, filed on Aug. 15, 2003, now Pat. No. 8,017,160.

(51) Int. Cl. A61K 31/205; A61P 3/02; A61K 9/48

(52) U.S. Cl. 424/451; 424/682

(57) ABSTRACT

Disclosed are methods and complexes for increasing magnesium uptake in mammals. Increasing magnesium uptake in mammals is accomplished by concurrently administering one or more magnesium salts with one or more quaternary amines and/or phosphatides and one or more di- or tri-carboxylic acids, in a one part dosage system or a multi-part dosage system. Increasing magnesium uptake in mammals may also be accomplished by concurrently administering one or more magnesium salts with an organic acid.

### CROSS REFERENCES TO RELATED APPLICATIONS

This application is a continuation-in-part (CIP) application of and claims the benefit of U.S. patent application Ser. No. 13/082,939, filed on Apr. 8, 2011, which is a continuation application of and claims the benefit of U.S. patent application Ser. No. 10/641,291, filed on Aug. 15, 2003, now U.S. Pat. No. 8,017,160, each of the contents of which are incorporated herein by reference in their entirety.

### FIELD

The present disclosure relates to increasing magnesium uptake in mammals. More particularly, the present disclosure relates to increasing magnesium uptake in mammals by administering one or more magnesium complexes.

### BACKGROUND

Magnesium is an important mineral in mammalian nutrition. As part of adenosine triphosphate (ATP), magnesium is used for all biosynthetic processes, glycolysis, formation of cyclic adenosine monophosphate (cyclic AMP), is involved in energy metabolism and energy dependent membrane transport and is used for ribonucleic acid (RNA) synthesis and transmission of the genetic code.

Magnesium, as a cation, is involved in over 10,000 enzyme (cell catalyst) actions. Magnesium is especially important to enzymes concerned with oxidative phosphorylation. Magnesium is also an important component of both intracellular and extracellular fluids. Intracellular magnesium is believed to control cellular metabolism by modulating the activity of rate limiting enzymes. Extracellular magnesium is important to the maintenance of electrical potentials of nerve and muscle membranes and for transmission of impulses across neuromuscular junctions. Magnesium has been shown to be important in maintaining the homeostasis of cardiac and smooth muscle tissues.

Magnesium is the fourth most common cation in the human body. For example, a typical human body contains on average 25 grams of magnesium. Approximately 59% of the magnesium is in the body's skeleton and bone structures, approximately 40% is in the body's musculature and soft body tissues and approximately 1% (about 2 to 2.8 grams) is in the body's extracellular fluid.

Considering that magnesium is an activator for so many important body functions, it is not surprising that deficiency can lead to a variety of serious physical and mental problems. Health conditions such as muscle spasms and tremors are associated with magnesium deficiency. In addition, nerve irritability, mood instability, high blood pressure (Essential, otherwise unexplained), angina (chest pain on exertion), heart arrythmias (magnesium is ‘nature's calcium channel blocker’) calcium loss/osteoporosis risk and insomnia are also associated with magnesium deficiency. Toxic metals (lead, mercury, cadmium, arsenic, and nickel) can accumulate more rapidly when magnesium stores are low and the replacement of magnesium in the body hastens elimination of toxic metals from the body.

Magnesium is inorganic and is not produced by the human body. Humans must rely upon dietary sources to provide the body with its magnesium requirements. Dietary magnesium intake has been declining in the United States, with a per capita decline of magnesium in the U.S. food supply (estimated as food flowing through the food distribution system) of from 408 mg/day in 1909 to 329 mg/day in 1986, almost a 20% decline. This decline has been attributed to the increase in consumption of processed foods. For example, ‘white foods’ such as refined flour, sugar, fat and processed or synthesized foods contain relatively little magnesium.

In those with normal digestion and assimilation, magnesium absorption from food is believed to be from approximately 40 to 60% of that ingested. However, there are many factors that can inhibit the body's ability to absorb magnesium. For example, phosphoric acid, which is present in most soft drinks, and oxalates in foods such as spinor and chocolate, combine with magnesium in the intestines and form insoluble compounds that are not absorbed by the body. Other factors that can reduce the function of the magnesium uptake system include: toxic minerals such as lead, mercury, arsenic, cadmium, and nickel; biocidal hormone mimics; metabolic cellular acidosis; phytates in ingested foods; caffeine intake; alcohol consumption; certain medications such as steroids and oral contraceptives; distress; enteropathy and other intestinal disorders; and maldigestion and digestive diseases.

Oral intakes of magnesium are difficult for the body to absorb. It is believed that only 3 to 12% of elemental magnesium, typically in the form of magnesium oxide, is absorbed for use by the body. Attempts have been made to make complexes to enhance the absorption of magnesium in the human body. For example, U.S. Pat. No. 5,849,337 to Dixon describes a complex containing magnesium, protein amino acids and ascorbic acid. As discussed above and in Dixon, a need exists for a method of increasing magnesium intake in mammals, particularly humans.

### SUMMARY

Magnesium complexes and methods of administering magnesium to a mammal are disclosed herein.

In an illustrative embodiment, the magnesium complex includes a magnesium salt, a quartemary amine or phosphatide and a di-carboxylic acid or tri-carboxylic acid. The complex may further include a solution of water and glycerol.

In a further illustrative embodiment, the magnesium salt is magnesium glycinate, magnesium ascorbate, magnesium chloride, magnesium sulfate, magnesium orotate, magnesium citrate, magnesium fumarate, magnesium malate, magnesium succinate, magnesium tartrate magnesium carbonate or similar magnesium salt. In another illustrative embodiment, the quartemary amine is choline and/or the phosphatide is phosphatidyl choline and the di- or tri-carboxylic acid is citrate.

In yet a further illustrative embodiment, the method for enhancing magnesium uptake in mammals includes administering to a mammal a magnesium salt, a quaternary amine or phosphatide and a di-carboxylic acid or tri-carboxylic acid. In one illustrative embodiment, the administering is oral and the mammal is human.

In another illustrative embodiment, the method of administering a magnesium complex according to the disclosure includes administering a two part system including a first dosage form and a second dosage form. The first dosage form may include one or more magnesium salts in equal proportions totaling about 650 mg of magnesium salts or about 110 mg of elemental magnesium. The second dosage form may include the quaternary amine or phosphatide, and the di-carboxylic acid or tri-carboxylic acid delivered as a solution. In one embodiment, the quaternary amine or phosphatide, and the di-carboxylic acid or tri-carboxylic acid includes about 750 mg of choline and about 550 mg of citrate in an about 50% vegetable glycerine and water solution. In an illustrative embodiment, the first and second dosage forms are administered concurrently. It is contemplated within the scope of the disclosure that the first and second dosage form can be administered separately.

In a further illustrative embodiment, the method according to the disclosure includes administering a medicament in a one part dosage system. In this illustrative embodiment, the medicament includes a softgel capsule containing one or more magnesium salts, for example, magnesium malate, in an amount sufficient to provide about 110 mg of elemental magnesium, phosphatidylcholine in an amount sufficient to provide about 500 mg of choline, and the MCTs oils as a carrier or fill solution. It is contemplated within the scope of the disclosure that other oral dosage forms may be used to administer the combination of magnesium complexes including tablets, capsules, caplets, solutions, suspensions, and/or syrups, and may also comprise a plurality of granules, beads, powders, mini-tablets, mini-soft gels or pellets that may or may not be encapsulated.

In another illustrative embodiment, the magnesium complex includes one or more magnesium salts and an organic acid. In an illustrative embodiment, the organic acid is taurine or 2-aminoethanesulfonic acid.

### BRIEF DESCRIPTION OF THE DRAWINGS

Embodiments of complexes and methods of use thereof are illustrated in the figures of the accompanying drawings which are meant to be exemplary and not limiting, in which like references are intended to refer to like or corresponding parts, and in which:

FIG. 1 illustrates a graph of the influence of choline citrate on ionized magnesium concentration in plasma.

### DETAILED DESCRIPTION

Detailed embodiments of magnesium complexes and methods of use thereof are disclosed herein, however, it is to be understood that the disclosed embodiments are merely exemplary of the complexes and methods, which may be embodied in various forms. Therefore, specific functional details disclosed herein are not to be interpreted as limiting, but merely as a basis for the claims and as a representative basis for teaching one skilled in the art to variously employ the present disclosure.

In an illustrative embodiment, magnesium complexes and methods of administering magnesium to a mammal are disclosed herein. It has been found that by administering to a mammal one or more magnesium salts, a quaternary amine or phosphatide and a di-carboxylic acid or tri-carboxylic acid, magnesium uptake in the subject could be improved.

The magnesium salts can be any known magnesium salts or a combination of several known magnesium salts. The magnesium salts can be mono magnesium or dimagnesium. In one illustrative embodiment, the magnesium salts include, magnesium glycinate, magnesium ascorbate, magnesium chloride, magnesium sulfate, magnesium orotate, magnesium citrate, magnesium fumarate, magnesium malate, magnesium succinate, magnesium tartrate, magnesium gluconate, and magnesium carbonate.

The quaternary amine or phosphatide can include any quaternary amine, phosphatide or a combination of quaternary amines and phosphatides. In one illustrative embodiment, the quaternary amine is choline.

The di-carboxylic acid or tri-carboxylic acid can include any di-carboxylic acid, tri-carboxylic acid or combination of di-carboxylic acids and tri-carboxylic acids. In an illustrative embodiment, the di-carboxylic acid is citrate.

In a further illustrative embodiment, the magnesium salt(s), the quaternary amine or phosphatide and the di-carboxylic acid or tri-carboxylic acid are administered concurrently. Concurrently, as used in herein, means administered within the same day. It is contemplated within the scope of the disclosure that the concurrently administration can be in a single daily dose or in divided doses administered during the course of the day. In an illustrative embodiment, they are administered together either as part of the same complex, which could include, for example a solution, capsule or tablet containing all of the components, or administered one after the other so that they can be metabolized together.

In an illustrative embodiment, the magnesium salt(s), the quaternary amine or phosphatide and the di-carboxylic acid or tri-carboxylic acid are administered as part of a complex in an aqueous glycerol or aqueous-glycerol solution. In another illustrative embodiment, the magnesium salt(s), the quaternary amine or phosphatide and the di-carboxylic acid or tri-carboxylic acid are administered as part of a complex in a solution containing both water and glycerol. In an illustrative embodiment, the solution contains approximately the same amount of water as glycerol. In one embodiment, the solution contains about 50% vegetable glycerine in water.

In a further illustrative embodiment, the magnesium salt(s), the quaternary amine or phosphatide, and the di-carboxylic acid or tri-carboxylic acid are administered in a multi-part dosage system. A fist dosage form may include the one or more magnesium salts as a powder delivered by a capsule. Various other delivery systems are known and can be used to administer the one or more magnesium salts, for example, tablets, pills, lozenges, pellets, capsules, capsules containing liquids, powders, granules, sustained-release formulations, suspensions (oily or aqueous), solutions, syrups, elixirs or any other form suitable for use.

Oral dosage forms are used to administer the combination of active agents, and include tablets, capsules, caplets, solutions, suspensions, and/or syrups, and may also comprise a plurality of granules, beads, powders, mini-tablets, mini-soft gels or pellets that may or may not be encapsulated.

Tablets may be manufactured using standard tablet processing procedures and equipment. One method for forming tablets is by direct compression of a powdered, crystalline, or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the magnesium salts, tablets prepared for oral administration using the method of the invention will generally contain other materials such as binders, diluents, lubricants, disintegrants, fillers, stabilizers, surfactants, coloring agents, and the like. Binders are used to impart cohesive qualities to a tablet, and thus ensure that the dosage form remains intact after compression. Suitable binder materials include, but are not limited to, starch (including corn starch and pre-gelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum.

Diluents are typically necessary to increase bulk so that a practical desirable size tablet is ultimately provided. Suitable diluents include dicalcium phosphate, calcium sulfate, lactose, cellulose, kaolin, mannitol, sodium chloride, dry starch, and powdered sugar. Lubricants are used to facilitate tablet manufacture; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers are used to inhibit or retard drug decomposition reactions that include, by way of example, oxidative reactions. Surfactants may be anionic, cationic, amphoteric, or nonionic surface active agents.

The dosage form may also be a capsule, in which case the magnesium salt containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. It is contemplated within the scope of the invention that tablets or mini-tablets may be placed inside a capsule along with other active ingredients.

In another illustrative embodiment, the one or more magnesium salts may include more than one of the magnesium salts in equal proportions totaling about 650 mg of magnesium salts or about 110 mg of elemental magnesium. It is contemplated within the scope of the disclosure that the magnesium salts may be in varying proportions totaling about 650 mg of magnesium salts.

A second dosage form may include the quaternary amine or phosphatide, and the di-carboxylic acid or tri-carboxylic acid delivered as a solution. In one embodiment, the quaternary amine or phosphatide, and the di-carboxylic acid or tri-carboxylic acid includes about 750 mg of choline and about 550 mg of citrate in about a 50% vegetable glycerine and water solution. In an illustrative embodiment, the first and second dosage forms are administered concurrently.

In another illustrative embodiment, the magnesium salt(s), and the quaternary amine or phosphatide are administered via a softgel capsule or shell in a one part dosage system in which one or more medium-chain triglycerides, (MCTs), oils are used as a carrier. The softgel formulation may include an outer layer of gelatin (made of gelatin, plasticizer, modifier, water, color, antioxidant or flavor). The outer layer can be enteric coated. In one embodiment, the softgel shell or outer layer includes an opacifier.

In general, the one or more MCTs are nonvolatile alkalinizing fatty acid esters of glycerol (e.g., medium-chain fatty acid esters of glycerol). Non-limiting examples of MCTs include fatty acid esters of glycerol in which the fatty acid moieties thereof have from about 4 to about 16 carbon atoms. The fatty acid moieties of the MCTs can be the same or different, and can be saturated or unsaturated.

In one embodiment, the magnesium salt(s), for example, magnesium malate, in an amount sufficient to provide about 110 mg of elemental magnesium, phosphatidylcholine in an amount sufficient to provide about 500 mg of choline, and the MCTs oils as a carrier or fill solution are disposed in the softgel capsule. It should be appreciated that the softgel capsule can have various shapes and sizes, depending on the design.

In another illustrative embodiment, the magnesium complex includes one or more magnesium salts and an organic acid, for improving magnesium uptake in a mammal. In an illustrative embodiment, the organic acid is taurine or 2-aminoethanesulfonic acid. The magnesium complex may also include one or more magnesium salts, an organic acid, and a di-carboxylic acid or tri-carboxylic acid.

In an illustrative embodiment, the magnesium salt(s) and the organic acid are administered concurrently for improving magnesium uptake in a mammal. In another illustrative embodiment, the magnesium salt(s) and the organic acid are administered as part of a complex in an aqueous glycerol or aqueous-glycerol solution. In yet another illustrative embodiment, the magnesium salt(s) and the organic acid are administered as part of a complex in a solution containing both water and glycerol. In an illustrative embodiment, the solution contains approximately the same amount of water as glycerol.

In a further illustrative embodiment, the magnesium complexes described herein may be used in the preparation of one or more medicaments for use in increasing magnesium uptake. In one embodiment, the medicament includes a softgel shell, one or more magnesium salts disposed within the softgel shell, a quaternary amine or phosphatide disposed within the softgel shell, and a medium-chain triglyceride oil carrier disposed within the softgel shell. In this embodiment, the softgel shell may include an opacifier. The one or more magnesium salts is magnesium malate present in an amount to provide about 110 mg of elemental magnesium. The quaternary amine or phosphatide is phosphatidylcholine present in an amount to provide about 500 mg of choline.

In other illustrative embodiments, the components of the magnesium complexes described herein may be mixed together into a single dosage form, for example in the form of a powder, capsule, capsule within capsule or tablet. Alternatively, the components of the magnesium complexes described herein may be present in one or more unit dosage forms of one or more of the components. Whether administered as a single unit dosage form comprising each of the individual components, or in two or more separate unit dosage form, the magnesium complexes described herein can be provided in the form of a kit for increasing magnesium uptake in a mammal.

In one embodiment, the kit may include a container including the one or more magnesium salts as a powder in the form of a capsule, the quaternary amine or phosphatide, the di-carboxylic acid or tri-carboxylic, the glycerine and water solution, and instructions for use. The instructions for use may include instructions for combining the quaternary amine or phosphatide and the di-carboxylic acid or tri-carboxylic with the glycerine and water solution.

In the kit according to the disclosure, the one or more magnesium salts, the quaternary amine or phosphatide, the di-carboxylic acid or tri-carboxylic, and the glycerine and water solution may each be pre-measured into a respective unit of use amount. In one embodiment, the magnesium salts are present in equal proportions totaling about 650 mg of magnesium salts or about 110 mg of elemental magnesium. The quaternary amine or phosphatide and the di-carboxylic acid or tri-carboxylic acid include about 750 mg of choline and about 550 mg of citrate, and the glycerine and water solution is about a 50% vegetable glycerine and water solution.

In illustrative embodiments, with reference to the following examples. subjects that had maladies associated with low magnesium levels were given a variety of combinations of compounds to determine their effectiveness.

The subjects in the examples were selected for signs of magnesium deficit including muscle irritability and fasciculation, benign cardiac irritability and/or moderate fibromyalgia pain that had previously been unresponsive to magnesium therapy.

### Example 1

A 26 year old man suffering from supraventricular arrythmias was unresponsive to medication and, despite detailed medical workup, was determined to be ‘idiopathic’.

Clinical assessments were done over periods of one month. Magnesium salts were administered to the subject for one month followed by one month of choline citrate alone followed by one month of magnesium salts along with concurrent administration of choline citrate.

During this time the patient was instructed to count the number of irregular heartbeats (IHB) over a 2 minute period on rising and before bed. The average number of irregular heart beat counted over the three month time period can be found in Table 1:

TABLE 1

| | AM | PM | Total |
|---|---|---|---|
| Month 1 (ave) | 13 | 22 | 35 |
| Month 2 (ave) | 11 | 27 | 38 |
| Month 3 (ave) | 1 | 3 | 4 |

As illustrated in Table 1, the average number of irregular heartbeats decreased drastically when the magnesium salts were administered along with concurrent administration of choline citrate.

### Example 2

A 72 year old woman suffering from ectopic heart beats which were unresponsive to medication and, despite detailed medical workup, was determined to be ‘idiopathic’.

Clinical assessments were done over periods of about one month. Magnesium salts were administered to the subject for one month followed by one month of choline citrate alone followed by one month of magnesium salts along with concurrent administration of choline citrate.

During this time the patient was instructed to count the number of irregular heartbeats (IHB) over a 2 minute period on rising and before bed. The average number of irregular heartbeats counted over the three month time period can be found in Table 2:

TABLE 2

| | AM | PM | Total |
|---|---|---|---|
| Month 1 (ave) | 18 | 20 | 38 |
| Month 2 (ave) | 22 | 19 | 41 |
| Month 3 (ave) | 1 | 2 | 3 |

As illustrated in Table 2, the average number of irregular heartbeats decreased drastically when the magnesium salts were administered along with concurrent administration of choline citrate.

### Example 3

A 59 year old man suffered from ‘restless legs’ and intermittent leg cramps during sleep. Clinical assessments were done with the subject taking magnesium salts for weeks 1 and 2, followed with the subject taking choline citrate alone for weeks 3 and 4, followed with the subject taking magnesium salts along with concurrent administration of choline citrate in weeks 5-9.

Each week the subject was asked to rate the intensity of the problem on a consistent rating scale with ‘0’ indicating no problem and ‘100’ indicating the most severe expression of the condition. The results can be found in Table 3.

TABLE 3

| | Ave. intensity of leg cramp |
|---|---|
| | related difficulty |
| Week 1-2 | 88 |
| Week 3-4 | 83 |
| Week 5 | 63 |
| Week 6 | 44 |
| Week 7 | 29 |
| Week 8 | 12 |
| Week 9 | 7 |

As illustrated in Table 3, the average intensity of the leg cramp related difficulty decreased drastically when the magnesium salts were administered along with concurrent administration of choline citrate.

The clinical assessments illustrated in Examples 1-3, are consistent with enhanced magnesium uptake when the correct magnesium salts are properly combined with high purity choline citrate.

### Example 4

Subjects were selected for signs of magnesium deficit including muscle irritability and fasciculation, benign cardiac irritability and/or moderate fibromyalgia pain that had previously been unresponsive to magnesium therapy.

The subjects were put on a fixed dose of magnesium salts, 2 capsules with each meal (6 per day) for one month. For the second month, the same regimen was continued plus the addition of 1 teaspoon of choline citrate in juice or water taken at the same time as the magnesium supplement. For the third month, the subject continued the magnesium regimen but without the addition of choline citrate. Diet and fluid intake were kept consistent during the study interval.

The amount of magnesium in each subject was measured at the end of each week using an ion specific electrode. The reference range for ionized magnesium using this ion specific electrode (NOVA) is 0.43-0.59 mmol/L. The results can be found below in Table 4.

TABLE 4

| | Subject: | | | |
|---|---|---|---|---|
| | Series #1 | Series #2 | Series #3 | Series #4 |
| | Ionized Mg | Ionized Mg | Ionized Mg | Ionized Mg |
| Results: | [plasma] | [plasma] | [plasma] | [plasma] |
| Initial Value | 0.31 mmol/L | 0.28 mmol/L | 0.33 mmol/L | 0.22 mmol/L |
| Week 1 | 0.30 | 0.27 | 0.30 | 0.25 |
| Week 2 | 0.32 | 0.23 | 0.35 | 0.24 |
| Week 3 | 0.30 | 0.20 | 0.31 | 0.22 |
| Week 4 | 0.33 | 0.22 | 0.29 | 0.23 |
| Week 5 | 0.39 | 0.30 | 0.37 | 0.26 |
| Week 6 | 0.44 | 0.37 | 0.44 | 0.33 |
| Week 7 | 0.47 | 0.48 | 0.49 | 0.39 |
| Week 8 | 0.51 | 0.52 | 0.55 | 0.44 |
| Week 9 | 0.50 | 0.49 | 0.52 | 0.42 |
| Week 10 | 0.47 | 0.45 | 0.47 | 0.39 |
| Week 11 | 0.45 | 0.47 | 0.45 | 0.40 |
| Week 12 | 0.42 | 0.40 | 0.39 | 0.41 |

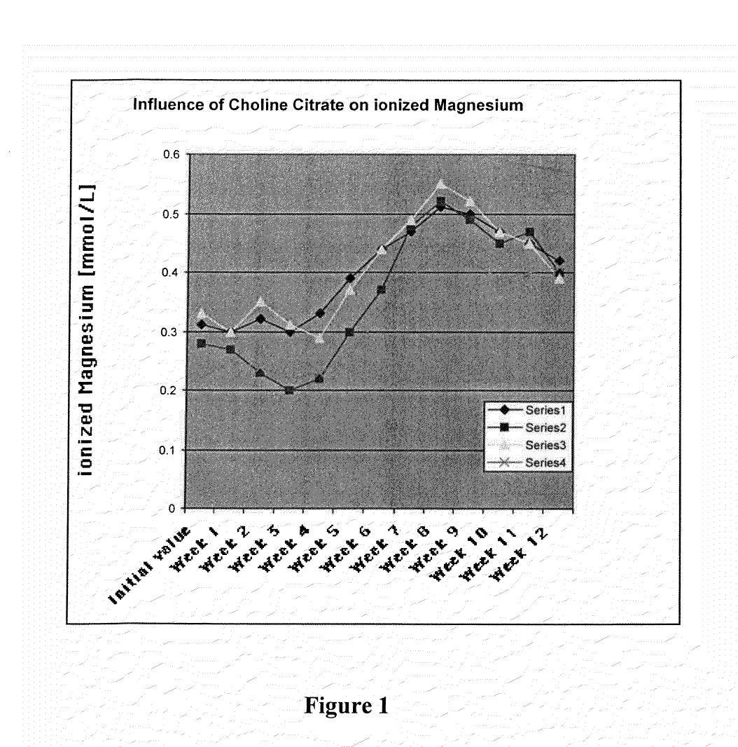

FIG. 1, is a graphical representation of the data of Table 4. As illustrated in FIG. 1 and Table 4, the amount of magnesium in the subject's plasma increased to its higher values following weeks 5-8 when the subjects were taking choline citrate along with the magnesium salts.

In other studies, the role of choline citrate on magnesium levels in the absence of magnesium supplementation was evaluated. A modest but statistically insignificant increase in magnesium was noted over a 30 day period of evaluation.

The examples illustrate that a previously unknown and unanticipated benefit is observed in the form of facilitated magnesium uptake when choline citrate is concurrently administered. Choline citrate alone does not substantially raise ionized magnesium levels. In some people with clinical magnesium need, even the most soluble and ionized forms of magnesium are less available, possibly because of inhibition of the Ca/Mg ATPase pump. A possible mechanism of action is the formation of micelles containing 2 molar equivalents of magnesium and choline along with 3 molar equivalents of citrate thus forming an electrically neutral complex.

The above description is presented to enable a person skilled in the art to make and use the complexes and methods disclosed herein, and is provided in the context of certain applications and requirements. Various modifications to the disclosed embodiments will likely be readily apparent to those skilled in the art, and the generic principles defined herein may be applied to other embodiments and applications without departing from the spirit and scope of the disclosure. Thus, this disclosure is not intended to be limited to the embodiments described herein, but is to be accorded the widest scope consistent with the principles and features disclosed herein. Therefore, the above description and examples should not be construed as limiting, but merely as exemplification of the various embodiments. Those skilled in the art will envision other modifications within the scope and spirit of the claims appended hereto.

What is claimed is:

1. A medicament, comprising:
one or more magnesium salts;
a quaternary amine or phosphatide;
a di-carboxylic acid or tri-carboxylic; and
a glycerine and water solution, wherein the quaternary amine or phosphatide and the di-carboxylic acid or tri-carboxylic are disposed in the glycerine and water solution.

2. The medicament of claim 1, wherein the one or more magnesium salts are present in an amount of about 650 mg.

3. The medicament of claim 1, wherein the quaternary amine or phosphatide is choline.

4. The medicament of claim 3, wherein the choline is present in an amount of about 750 mg.

5. The medicament of claim 1, wherein the di-carboxylic acid or tri-carboxylic acid is citrate.

6. The medicament of claim 5, wherein the citrate is present in an amount of about 550 mg.

7. The medicament of claim 1, wherein the glycerine and water solution is a 50% vegetable glycerine and water solution.

8. The medicament of claim 1, wherein the one or more magnesium salts are a powder adapted to be delivered by a capsule.

9. A medicament, comprising:
a softgel shell;
a magnesium salt disposed within the softgel shell;
a quaternary amine or phosphatide disposed within the softgel shell; and
a medium-chain triglyceride oil disposed within the softgel shell.

10. The medicament of claim 9, wherein the softgel shell includes an opacifier.

11. The medicament of claim 9, wherein the magnesium salt is magnesium malate.

12. The medicament of claim 11, wherein the magnesium malate is present in an amount to provide about 110 mg of elemental magnesium.

13. The medicament of claim 9, wherein the quaternary amine or phosphatide is phosphatidylcholine.

14. The medicament of claim 13, wherein the phosphatidylcholine is present in an amount to provide about 500 mg of choline.

15. A medicament, comprising:
one or more magnesium salts; and
an organic acid.

16. The medicament of claim 15, wherein the organic acid is taurine.

17. The medicament of claim 16, further comprising a di-carboxylic acid or tri-carboxylic acid.
